(19) United States
(12) Patent Application Publication
(10) Pub. No.: US 2026/0193154 A1
(43) Pub. Date: Jul. 9, 2026

(54) PROCESS AND COMPOSITION FOR CHEMICAL RECYCLING OF POLYMERS HAVING REDUCED OXYGEN CONTENT

(71) Applicant: SABIC GLOBAL TECHNOLOGIES B.V., Bergen op Zoom (NL)

(72) Inventors: Maria SOLIMAN, Selfkant (DE); Nicolas GOYHENEIX, Riemst (BE); Carolus WILSENS, Veldhoven (NL); Johan PASTWA, Elsloo (NL); Patrick Elisabeth Luc VOETS, Born (NL); Gerardus Theodorus Cornelis KWAKKENBOS, Meerssen (NL); Safa FARAJZADEH BIBALAN, Eindhoven (NL)

(21) Appl. No.: 19/132,557

(22) PCT Filed: Nov. 17, 2023
(86) PCT No.: PCT/EP2023/082172

§ 371 Date: May 23, 2025

(30) Foreign Application Priority Data
Nov. 28, 2022 (EP) 22209970.7

(51) Int. Cl. C07C 4/04

(52) U.S. Cl. CPC C07C 4/04

(57) ABSTRACT

A process for chemical recycling, wherein the process involves the steps of:
i. supplying a composition of polymers;
ii. subjecting the composition of polymers to a thermal treatment to obtain a pyrolysis oil;
iii. optionally, subjecting the product obtained in step ii. to a hydrotreatment process;
iv. subjecting the product obtained in step ii., or, when applied, the product obtained in step iii. to a thermal decomposition process, to obtain a chemical composition including ethylene and propylene;

wherein the composition of polymers comprises at most 200 ppm by weight of oxygen atoms, with regard to the total weight of the composition of polymers; and
wherein the composition of polymers includes polyolefin materials including one or more stabilisation compound(s), wherein the stabilisation compound(s) are selected from compounds including ≤20.0 wt % of oxygen atoms, with regard to the total weight of the compound.

### CROSS REFERENCE TO RELATED APPLICATIONS

This application is a National Stage Application of PCT/EP2023/082172, filed Nov. 17, 2023, which claims the benefit of European Application No. 22209970.7, filed Nov. 28, 2022, both of which are incorporated by reference in their entirety herein.

### BACKGROUND

The present invention relates to a process for chemical recycling of polymers.

The invention further relates to a composition of polymers that is suitable for use in chemical recycling processes, in particular in chemical recycling processes involving pyrolysis of polymer compositions to obtain pyrolysis oils and subsequent processing of such pyrolysis oils via steam cracking or via refinery operations to obtain chemical feed streams for the production of polymers.

In order to mitigate the end-of-life issues that relate to polymer materials, such as thermoplastics, there is as increasingly stringent drive to seek to for applications wherein polymer materials that are no longer considered to be of use for their original purpose can be utilised in a meaningful way, while minimising the burden on the environment, such as by discarding the materials as landfill or incineration.

To achieve such, a vast array of recycling solutions for materials has been and is being investigated. As part of such investigations, a particular aspect that is paramount to be considered is the chemical composition of the polymer materials that one has available for processing. Typically, streams of polymer materials that one can access contain a wide variety of polymers of different chemical constitution, due to the fact that such streams are typically collected in a combined way; at the user level, e.g. the household, the knowledge and means that would allow the user to separate one type of polymer from the other generally is not available, nor can likely be expected to be available. And whilst there are certain plastics sorting technologies available, and increasingly developed, at present there remains the situation that the vast majority of waste plastics streams de facto are comprising polymer materials of differing chemical nature—and it is not foreseeable that this is likely to change.

Accordingly, it is appreciated that compositions of waste polymers of varying chemical nature can be processed via routes of desirably high value and desirably low environmental impact.

A particular route via which mixed streams of waste polymers can be processed that is gaining traction is via chemical recycling routes. Such routes typically involve a first stage of processing waste polymer streams of certain, defined, composition to produce one or more chemical compositions of oily nature, for example compositions that would be comparable to naphtha-type compositions as one can obtain from refining fossil crude oils, which stage then may be followed by the processing of such oily compositions via thermochemical decomposition processes to obtain hydrocarbon chemical compositions comprising a slate of chemicals that can be used again for manufacturing new, or ‘virgin’, products, including ‘virgin’ polymer materials such as for example polyethylenes and polypropylenes.

Such chemical recycling routes can be considered as a (part of) a solution for dealing with the abundantly available waste plastics streams. However, the composition of the waste plastics streams can affect the efficiency of the operation of such chemical recycling routes.

A particular element that may be detrimental to the capability of processing waste plastics via chemical recycling routes is oxygen. The presence of oxygen, both in element form and in chemical compounds, may lead to corrosion of equipment that is employed in unit operations for production of chemical compositions comprising ethylene and propylene, such as in steam cracking operations.

To mitigate this issue, processes of the art provide for separate unit operations to capture oxygen-containing compounds at various stages of the chemical recycling process. Such stages may include hydrotreatment stages, and polishing stages. As will be understood, in view of process efficiency it is preferred to minimise additional stages that need to be used in such chemical recycling process; each stage comes at a cost, involves additional consumption of energy, and leads to reduction of the yield of the process. Therefore, it is desired to employ a process in which the least amount of unit operations or process stages have to be incorporated.

### SUMMARY

The inventors of the present application have now found a particularly suitable process for chemical recycling, wherein the process involves the steps of:
i. supplying a composition of polymers;
ii. subjecting the composition of polymers to a thermal treatment to obtain a pyrolysis oil;
iii. optionally, subjecting the product obtained in step ii. to a hydrotreatment process;
iv. subjecting the product obtained in step ii., or, when applied, the product obtained in step iii. to a thermal decomposition process, to obtain a chemical composition comprising ethylene and propylene;
   wherein the composition of polymers comprises at most 200 ppm, preferably at most 150 ppm, more preferably at most 100 ppm, even more preferably at most 50 ppm by weight of oxygen atoms, with regard to the total weight of the composition of polymers; and
   wherein the composition of polymers comprises polyolefin materials comprising one or more stabilisation compound(s), wherein the stabilisation compound(s) are selected from compounds comprising ≤20.0 wt % of oxygen atoms, preferably ≤15.0 wt % of oxygen atoms, more preferably ≤10.0 wt %, with regard to the total weight of the compound.

### DETAILED DESCRIPTION

Such process allows for efficient chemical recycling of polymers, using a simplified process, wherein the corrosion of the equipment employed in the process is reduced. For example in operations wherein the chemical recycling involves steam cracking, such process allows for the use of an increased fraction of the product of step ii., e.g. a pyrolysis oil product, and/or the product of step iii, e.g. a hydrotreated pyrolysis oil product, without detrimental effects on the steam cracking process, such as corrosion, reduction of service life of the steam cracker, or fouling.

It is preferred that the stabilisation compound(s) are selected from
tocopherols, preferably α-tocopherol;
dialkyl hydroxyl amines, preferably distearyl hydroxyl amine; and
bisalkyl methyl amine oxides, preferably bis(hydrogenated rapeseed oil alkyl) methyl amine oxides.

For example, the composition of polymers may comprise ≤2000 ppm by weight of the stabilisation compound(s), preferably ≥100 and ≤2000 ppm, more preferably ≥500 and ≤2000 ppm, with regard to the total weight of the composition of polymers.

Preferably, the composition of polymers comprises ≥1 ppb by weight of oxygen atoms, with regard to the total weight of the composition of polymers, preferably ≥5 ppb, more preferably ≥10 ppb.

Preferably, the composition of polymers comprises ≤40 ppm by weight of oxygen atoms, more preferably ≤30 ppm, even more preferably ≤20 ppm.

The thermal treatment of step ii. may for example involve a low-severity pyrolysis process, wherein the pyrolysis of the composition of polymers is performed at a temperature of ≥250° C. and ≤450° C., or a high-severity pyrolysis process, wherein the pyrolysis of the composition of polymers is performed at a temperature of >450° C. and ≤650° C.

Alternatively, the thermal treatment may be a catalytic process, preferably wherein the thermal treatment is a process operated in the presence of a ZSM-5 zeolite catalyst and/or a spent FCC catalyst.

In the process of the present invention, the hydrotreatment step iii. may be performed at a temperature of ≤350° C., in the presence of hydrogen, preferably at a pressure of ≤10.0 MPa, preferably ≥1.0 and ≤10.0 MPa, more preferable at ≥2.0 and ≤7.0 MPa.

For example, the hydrotreatment step iii. may be performed in the presence of a catalyst, wherein the catalyst is selected from a cobalt-molybdenum catalyst on alumina support, a nickel-molybdenum catalyst on alumina support, a tungsten-molybdenum catalyst on alumina support, a platinum-palladium catalyst on alumina support, a nickel sulphide catalyst, a molybdenum sulphide catalyst, or a nickel-molybdenum sulphide catalyst.

The thermal decomposition process of step iv. may be a steam cracking process, preferably wherein the steam cracking occurs in a steam cracking unit comprising heated coils, wherein the coil outlet temperature (COT) is in the range of 800° C. to 870° C. Alternatively, the thermal decomposition process of step iv. may be a catalytic cracking process.

In the embodiment wherein the thermal decomposition process of step iv. is a steam cracking process, the feed composition that is supplied to the steam cracking process may for example comprise ≥2.5 wt % and ≤75.0 wt %, preferably ≥5.0 wt % and ≤50.0 wt %, more preferably >10.0 wt % and ≤50.0 wt % of the product obtained in step ii.

Alternatively, the feed composition that is supplied to the steam cracking process may for example comprise ≥2.5 wt % and ≤75.0 wt %, preferably ≥5.0 wt % and ≤50.0 wt %, more preferably ≥10.0 wt % and ≤50.0 wt % of the product obtained in step iii.

In the process of the present invention, the composition of polymers may for example comprise:
≥70.0 wt % of polyolefin compositions; and
≤200 ppm by weight of oxygen atoms, preferably ≥1 ppb and ≤150 ppm, more preferably ≥10 ppb and ≤100 ppm, even more preferably ≥10 ppb and ≤50 ppm;
   with regard to the total weight of the composition of polymers.

The invention also relates to a composition of polymers comprising:
≥70.0 wt % of polyolefin compositions; and
≤200 ppm by weight of oxygen atoms, preferably ≥1 ppb and ≤150 ppm, more preferably ≥10 ppb and ≤100 ppm, even more preferably ≥10 ppb and ≤50 ppm;
   with regard to the total weight of the composition of polymers.

The composition of polymers may for example comprise one or more stabilisation compound(s), the stabilisation compound(s) are selected from tocopherols, preferably wherein the stabilisation compound(s) include α-tocopherol.

The composition of polymers may for example comprise one or more stabilisation compound(s), wherein the stabilisation compound(s) are selected from compounds comprising ≤20.0 wt % of oxygen atoms, preferably ≤15.0 wt % of oxygen atoms, more preferably ≤10.0 wt %, with regard to the total weight of the compound.

It is particularly preferred that the composition of polymers is obtained as a waste plastics stream, for example from post-consumer or household wastes.

The invention also relates to the use of a composition of polymers according to the invention for the reduction of fouling and/or corrosion during steam cracking of chemical feeds comprising waste plastics-derived materials.

Such process allows for efficient chemical recycling of polymers, using a simplified process, wherein the corrosion of the equipment employed in the process is reduced.

The hydrotreatment process of step iii. may for example be performed in one of more vessel(s) configured to hold a hydrotreatment catalyst. The vessel may be configured to operate in gas phase, liquid phase, vapour-liquid phase, or slurry phase.

The vessel may include one or more beds of the hydrotreatment catalyst. Such bed(s) may be fixed bed(s), fluidized bed(s), moving bed(s), slurry bed(s), or combinations thereof. The vessel may be operated in adiabatic, isothermal, non-adiabatic, or non-isothermal conditions.

In the hydrotreatment step, the product of step ii. may be subjected to treatment in the presence of hydrogen, wherein the volume flow ratio of hydrogen to the product of step ii. may for example be 10 to 3000, preferably 200 to 1000.

The hydrotreatment step iii may be performed in the presence of a catalyst. Such catalyst may for example be a catalyst selected from a cobalt-molybdenum catalyst on alumina support, a nickel-molybdenum catalyst on alumina support, a tungsten-molybdenum catalyst on alumina support, a platinum-palladium catalyst on alumina support, a nickel sulphide catalyst, a molybdenum sulphide catalyst, or a nickel-molybdenum sulphide catalyst.

The catalyst that may be used in the hydrotreatment step iii. may for example be sulphided.

The composition of polymers preferably comprises ≥70.0 wt % of polyolefins.

Such polyolefins preferably comprise polyethylenes and polypropylenes. In particular, the polyolefins may comprise ≥80.0 wt % of polyethylenes, or ≥90.0 wt % of polyethylenes. The polyolefins may comprise ≤20.0 wt % of polypropylenes, or ≤10.0 wt % of polypropylenes.

Such polyethylenes may be a composition comprising low-density polyethylenes, linear low-density polyethylenes, and high-density polyethylenes.

It is particularly preferred that the composition of polymers comprises a high fraction of polyolefins, such as ≥70.0 wt %, or ≥80.0 wt %, or ≥90.0 wt %, with regard to the total weight of the composition of polymers. Compositions of polymers comprising such high fraction of polyolefins are particularly suitable for chemical recycling via catalytic or non-catalytic thermal treatment processes, due to the fact that their polymer structure is based on linear monomers, which are suitable for thermal cracking.

Via such thermal cracking, for example steam cracking or catalytic cracking, the composition of plastics, in particular a composition of waste plastics, may be converted into chemical building blocks, in particular ethylene and propylene.

Accordingly, the process of the present invention allows for the suitable conversion of waste plastics into new plastics of high quality, thereby creating a circular economy of material use.

1. A process for chemical recycling of polymers, the process involving the steps of:
i. supplying a composition of polymers;
ii. subjecting the composition of polymers to a thermal treatment to obtain a pyrolysis oil;
iii. optionally, subjecting the product obtained in step ii. to a hydrotreatment process;
iv. subjecting the product obtained in step ii., or, when applied, the product obtained in step iii. to a thermal decomposition process, to obtain a chemical composition comprising ethylene and propylene;
wherein the composition of polymers comprises at most 200 ppm by weight of oxygen atoms, with regard to the total weight of the composition of polymers; and
wherein the composition of polymers comprises polyolefin materials comprising one or more stabilisation compound(s), wherein the stabilisation compound(s) are selected from compounds comprising ≤20.0 wt % of oxygen atoms, with regard to the total weight of the compound.

2. The process according to claim 1, wherein the stabilisation compound(s) are selected from
tocopherols;
dialkyl hydroxyl amines; and
bisalkyl methyl amine oxides.

3. The process according to claim 1, the composition of polymers comprises ≥1 ppb by weight of oxygen atoms, with regard to the total weight of the composition of polymers.

4. The process according to claim 1,
wherein the thermal treatment of step ii. involves a low-severity pyrolysis process, wherein the pyrolysis of the composition of polymers is performed at a temperature of ≥250 C. and ≤450° C., or a high-severity pyrolysis process, wherein the pyrolysis of the composition of polymers is performed at a temperature of >450° C. and ≤650° C.

5. The process according to claim 1, wherein the thermal treatment is a catalytic process.

6. The process according to claim 1, wherein the hydrotreatment step iii. is performed at a temperature of ≤350° C., in the presence of hydrogen.

7. The process according to claim 1, wherein the hydrotreatment step iii. is performed in the presence of a catalyst, wherein the catalyst is selected from a cobalt-molybdenum catalyst on alumina support, a nickel-molybdenum catalyst on alumina support, a tungsten-molybdenum catalyst on alumina support, a platinum-palladium catalyst on alumina support, a nickel sulphide catalyst, a molybdenum sulphide catalyst, or a nickel-molybdenum sulphide catalyst.

8. The process according to claim 1, wherein the thermal decomposition process of step iv. is a steam cracking process or wherein the thermal decomposition process of step iv. is a catalytic cracking process.

9. The process according to claim 8, wherein the feed composition that is supplied to the steam cracking process comprises ≥2.5 wt % and ≤75.0 wt % of the product obtained in step ii;
and/or
wherein the feed composition that is supplied to the steam cracking process comprises ≥2.5 wt % and ≤75.0 wt % of the product obtained in step iii.

10. The process according to claim 1, wherein the composition of polymers comprises:
≥70.0 wt % of polyolefin compositions; and
≤200 ppm by weight of oxygen atoms;
with regard to the total weight of the composition of polymers.

11. A composition of polymers comprising:
≥70.0 wt % of polyolefin compositions; and
≤200 ppm by weight of oxygen atoms;
with regard to the total weight of the composition of polymers.

12. The composition according to claim 11, wherein the composition of polymers comprises one or more stabilisation compound(s), wherein the stabilisation compound(s) are selected from compounds comprising ≤20.0 wt % of oxygen atoms, with regard to the total weight of the compound.

13. Composition according to claim 12, wherein the stabilisation compound(s) are selected from tocopherols.

14. The composition according to claim 11, wherein the composition is obtained as a waste plastics stream from post-consumer wastes.

15. A method for reducing fouling and/or corrosion during steam cracking of chemical feeds comprising waste plastics-derived materials, the method comprising steam cracking the composition of polymers according to claim 11.
